(19) United States
(12) Patent Application Publication
(10) Pub. No.: US 2005/0209205 A1
(43) Pub. Date: Sep. 22, 2005

(54) Enhancement of anti-androgenic activity by a combination of inhibitors targeting different steps of a steroid-dependent gene activation pathway and uses thereof

(76) Inventors: Charles Shih, San Diego, CA (US); Ching-Yuan Su, San Diego, CA (US)

Correspondence Address: DAVID R PRESTON & ASSOCIATES APC, 12625 HIGH BLUFF DRIVE, SUITE 205, SAN DIEGO, CA, 92130, US

(21) Appl. No.: 11/045,181

(22) Filed: Jan. 27, 2005

Related U.S. Application Data
(60) Provisional application No. 60/539,753, filed on Jan. 28, 2004.

(51) Int. Cl. A61K 31/56; A61K 31/12

(52) U.S. Cl. 514/171; 514/685

(57) ABSTRACT

The present invention includes novel methods and compositions for inhibiting or reducing steroid-dependent gene activation including the administration of at least two compounds that act different steps within a steroid receptor gene activation pathway. Preferred methods may include administering a first compound able to induce degradation of a steroid receptor and administering a second compound able to inhibit gene activation at a different step of the steroid receptor pathway. Steroid-dependent gene activation may be reduced or inhibited, modulated or controlled greater when the at least two of the compounds are administered in combination or together.

### CROSS REFERENCE TO RELATED APPLICATIONS

The present application claims benefit of priority to U.S. Provisional Patent Application Ser. No. 60/539,753, filed on Jan. 28, 2004, entitled, “Enhancement of Antiandrogenic Activity By Combination of Inhibitors Targeted at Different Steps of Androgen-Induced AR-Activation Pathway and Uses Thereof,” and is herein incorporated by referenced in its entirety.

### TECHNICAL FIELD

The present invention relates generally methods and compositions for the treatment of steroid-related medical conditions. More specifically, the present invention includes methods and compositions capable of inhibiting or reducing steroid-dependent gene activation by administering at least two compounds capable of targeting different steps of the steroid-induced gene activation pathway.

### BACKGROUND OF THE INVENTION

The androgen receptor (AR) is a member of the steroid receptor superfamily. Steroid receptors act as transcription factors when bound to their cognate hormone ligands. Steroid receptors typically include a ligand binding doman (LBD), a hinge region and a DNA binding domain (DBD).

Androgen exerts its function by entering a target cell and binding to a specific androgen receptor (AR), leading to the activation of androgen-regulating genes. The male circulating androgen hormone, testosterone, is converted within cells of peripheral tissues by 5-alpha reductase enzymes to the main intracellular androgen, dihydrotestosterone (DHT). Pharmaceutical companies have developed drugs that either inhibit the conversion of testosterone into DHT or that interfere with the binding between androgen and AR. For example, the 5-alpha-reductase inhibitor, finasteride (Prosca and Propecia), and the androgen-AR binding inhibitors, flutamide and bicalutamide (Casodex), are non-steroid anti-androgens that have been used to treat benign prostate hyperplasia, baldness, prostate cancer and other androgen disorders. However, these anti-androgen drugs can cause side effects, such as impotence in some men receiving treatment. This is mainly due to the non-discriminatory inhibition of androgen activity by these types of anti-androgen.

The androgen-induced AR activation pathway is a multiple step process (see, for example, Lee and Chang (2003) J. Clin. Endocrinol. Metab., 88: 4043-4054, which is incorporated by reference in its entirety herein) and does not simply involve androgen and AR binding. The AR activation pathway may be summarized by the following steps:
1) AR Expression: Androgen target (androgen-responsive) cells, which carry the AR gene in their nucleus, express AR proteins by transcribing the AR gene (DNA) into AR messenger RNA (AR mRNA). AR mRNA moves into the cytoplasm where AR synthesis occurs. The synthesized AR proteins form complexes with chaperone proteins (heat shock proteins, such as hsp70 and hsp90), which stabilize the AR proteins and help maintain their affinity for their ligand, androgen.
2) Intracellular Androgen Transformation: Testosterone from circulation enters the target cells and is converted by the enzymes 5-alpha-reductases I and II into the main intracellular androgen, dihydrotestosterone (DHT).
3) Androgen Receptor Activation, Dimerization, and Localization: Cytoplasmic androgen (DHT) binds to AR, which then dissociates from chaperone proteins to form androgen-AR complexes that are then translocated into the nucleus. During the binding and translocation process, the androgen-AR complexes are phosphorylated by protein kinase and undergo dimerization
4) Interaction with AR Co-Regulators: Dimerized AR proteins may also interact with other regulatory proteins, that is to say, AR co-regulators or androgen receptor associated proteins (ARA), which can up- or down-regulate AR activity.
5) Androgen Response Element Binding: Within the nucleus, the androgen-AR-ARA complexes, functioning as a transcription factor, bind to the androgen response element (ARE) on the promoter region of androgen-regulated gene(s) and recruit other regulatory proteins involved in general transcription machinery that lead to the activation (expression or repression) of androgen-responsive target gene(s).

### SUMMARY

The present invention includes novel methods and compositions for inhibiting or reducing steroid-dependent gene activation including the administration of at least two compounds. The compounds may be administered to a biological sample such as a eukaryotic cell or an organism such as a human. The first compound is capable of inducing degradation of a steroid receptor and the second compound is capable of inhibiting or reducing gene activation by the steroid receptor. The second compound may act at a different step along the steroid receptor gene activation pathway. The second compound may inhibit binding between a steroid and a steroid receptor, a steroid receptor binding to a steroid response element, a steroid receptor binding to a steroid receptor associated protein or cofactor, nuclear transfer of a steroid receptor, transcription of a steroid receptor, or translation of a steroid receptor. Steroid-dependent gene activation may be reduced or inhibited greater when at least two of the compounds are administered in combination or together than when administering a single compound.

In another aspect of the present invention a composition is disclosed including a first compound capable of inducing degradation of a steroid receptor and a second compound capable of inhibiting the steroid receptor from activating a gene. The first and second compounds act at different points or steps within a steroid receptor pathway.

In another aspect of the present invention a pharmaceutical composition is disclosed including a first compound or pharmaceutically acceptable salt thereof able to induce degradation of a steroid receptor, a second compound or a pharmaceutically acceptable salt thereof able to inhibit steroid-dependent gene activation, and a pharmaceutically acceptable diluent, adjuvant or carrier. The first compound and the second compound act at different steps of a steroid receptor pathway and, when in combination, are provided in a therapeutically effective amount. When the steroid receptor is an androgen receptor, examples of potential compounds include but are not limited to curcumin derivatives or analogues (such as but not limited to ASCJ-9 and ASCJ-15), bicalutamide, hydroxyflutamide, docosahexaenoic acid (DHA), silibinin (SB), quercetin (QU), finesteride, dutasteride, or a functional derivative thereof.

In another aspect of the present invention a method of preventing or treating a steroid modulated medical condition in a human is disclosed including providing an individual suspected of suffering from a steroid modulated medical condition, and administering a therapeutically effective amount of at least one of the disclosed pharmaceutical compositions. The pharmaceutical compositions include at least two compounds capable of reducing or inhibiting steroid-dependent gene activation and act at different steps of a steroid receptor pathway. Preferably the at least two compounds reduce or inhibit steroid-dependent gene activation to a greater degree when administered in combination or together than when administered alone. Examples of medical conditions that may benefit from the present invention include but are not limited to acne, hirsutism, androgenetic alopecia (male pattern baldness), benign prostate hyperplasia, prostate cancer, bladder cancer, lung cancer, liver cancer, breast cancer and cervical cancer.

In other aspects of the present invention methods and compositions for wound or inflammation treatment are disclosed, the methods may include topically administering one or more of the disclosed compositions, pharmaceuticals or cosmetics to an individual desiring treatment of a wound or inflammation.

In other aspects of the present invention, methods of administering the disclosed compositions or pharmaceuticals in a human are provided for the treatment, inhibition or reduction of medical conditions relating to steroid-dependent gene activation. The methods may include encapsulating a composition or pharmaceutical in a liposome and administering the encapsulated composition or pharmaceutical to the desired individual.

### BRIEF DESCRIPTION OF THE FIGURES

FIG. 1 depicts a Western Gel image from lysed human prostate carcinoma cells, LNCaP cells, stained with Coomassie Blue demonstrating a decrease in the endogenous level of the androgen receptor (AR) when administering 1 μM of a curcumin derived compound, termed ASCJ-15. The hormone by product of testosterone, dihydrotestosterone (DHT), or a vehicle control was added to the cultured LNCaP cells according to Example 1 at 2 nM for 24 hours. The relative density of the androgen receptor (AR) is also depicted with DHT or with control vehicle and was derived by normalizing the signal of the androgen receptor (AR) signal with that of actin.

FIG. 2 depicts a Western Gel image from lysed human prostate carcinoma cells, LNCaP cells, stained with Coomassie Blue demonstrating degradation of the androgen receptor the presence of 1 μM of a curcumin derived compound, the particular compound termed ASCJ-15. Specifically Lanes 1, 4, 7 and 10 correspond to LNCaP cultured cells; Lanes 2, 5, 8 and 11 correspond to cultured LNCaP cells treated with 1 μM of ASCJ-15, a curcumin derivative; and Lanes 3, 6, 9 and 12 correspond to cultured LNCaP cells treated with a negative control, vitamin E. The time period positioned above the referenced lanes correspond to the time after which cyclohexamide, a protein synthesis inhibitor, was added to the culture. β-actin was provided as a control demonstrating overall protein was not degraded.

FIG. 3 depicts a graphical representation of the inhibitory effect of ASCJ-15, a curcumin derivative, and decosahexanoic acid (DHA) on androgen-induced gene activation in human prostate carcinoma cells, LNCaP cells. The androgen receptor (AR) transactivation assay was conducted according to Example 1 using the concentrations of inhibitors depicted in the figure. While DHA and ASCJ-15 alone were able to suppress DHT-induced transactivation, DHA and ASCJ-15 in combination demonstrated the greatest inhibition of DHT induced androgen receptor (AR) activity.

FIG. 4 depicts a graphical representation of the inhibitory effect of ASCJ-15 and DHA on androgen-induced human prostate carcinoma cell growth. ASCJ-15 and DHA alone were capable of suppressing LNCaP cell growth greater than the vehicle control. However, the greatest suppression of LNCaP cell growth was found when administering ASCJ-15 and DHA in combination.

FIG. 5 depicts a graphical representation of the inhibitory effect of hydroxyflutamide (HF), a metabolite of flutamide, and decosahexanoic acid (DHA), alone and in combination, on androgen-induced gene activation in human prostate carcinoma cells, LNCaP cells. While HF and DHA were able to suppress the DHT-induced androgen receptor (AR) activity, the greatest suppression of DHT-induced activity was observed when HF and DHA were used in combination.

FIG. 6 depicts a graphical representation of the inhibitory effect of ASCJ-15 and Silibinin (SB) alone and in combination on androgen-induced gene activation in human prostate carcinoma cells, LNCaP cells. ASCJ-15 and SB were able to significantly suppress DHT-induced androgen receptor (AR) activity. When administered together ASCJ-15 and SB had an additive effect of the suppression on DHT-induced gene activation.

FIG. 7 depicts a graphical representation of the inhibitory effect of ASCJ-15 and SB on androgen-induced LNCaP cell growth. Significant suppression of DHT-induced cell growth was observed when ASCJ-15 and SB were administered alone. When administered in combination, ASCJ-15 and SB demonstrated an additive effect on the suppression of DHT-induced LNCaP cell growth.

FIG. 8 depicts a graphical representation of the inhibitory effect of ASCJ-15 and Quercetin (QU) alone and in combination on androgen-induced gene activation in human prostate carcinoma cells, LNCaP cells. Significant suppression of DHT-induced androgen receptor (AR) activity was observed when ASCJ-15 and QU were administered alone. A synergistic relationship was observed when ASCJ-15 and QU were administered in combination.

FIG. 9 depicts a graphical representation of the inhibitory effect of hydroxyflutamide (HF), a metabolite of flutamide, and ASCJ15 alone and in combination on wild-type androgen-induced gene activation. ASCJ15 and HF were able to significantly suppress wild-type DHT-induced androgen receptor (AR) activity. When administered together ASCJ15 and SB had an additive effect of the suppression of DHT-induced gene activation.

### DETAILED DESCRIPTION

The present invention includes methods and compositions for the regulation of steroid-dependent gene activation. The methods and compositions setforth in the present invention include the use of at least two compounds, each capable of modulating, inhibiting, suppressing or reducing the effect or activation associated with a steroid receptor. The utilization of two compounds acting at different steps along the steroid receptor gene activation pathway may allow additional regulation or suppression of the effect or activity of a steroid receptor. The utilization of two compounds may provide increased suppression, regulation or modulation as compared to a single compound used independently or administered alone.

I. Methods of Inhibiting or Reducing Steroid-dependent Gene Activation using a Combination of Inhibitors

The present invention includes methods of modulating or reducing the effect of a steroid receptor using a combination of inhibitors. Steroid receptors such as the androgen receptor (AR), progesterone receptor (PR), glucocorticoid receptor (GR), thyroid receptor, peroxisome proliferator-activated receptor (PPAR), retinoid X receptors (RXR), and orphan steroid receptors and the like regulate gene transcription by a series of events that begin with the binding between a steroid and the steroid receptor and result in gene activation via interaction between the steroid receptor and a corresponding steroid response element (SRE). More specifically, a steroid receptor gene regulation or activation pathway may include a steroid binding the steroid receptor, dimerization of the steroid receptor (optionally to form a homodimer), translocation of the steroid receptor to the nucleus, the steroid receptor binding to a cofactor (such as ARA), binding of the steroid receptor to the appropriate steroid response element and optionally activation of other transcription factors. By administering at least two compounds that act along the steroid receptor pathway the present invention provides a potential therapeutic treatment for a variety of conditions. Preferably the at least two compounds act at different steps along the steroid receptor pathway such that when administered together or administered in cooperation the compounds are able to modulate the effect of the steroid receptor at two points. This may provide greater control or suppression of steroid-dependent gene regulation and therefore may provide related benefits in the treatment of a disease or a steroid related condition.

The method may be applied to biological samples including isolated cells, such as eukaryotic cells grown in in vitro culture for assay purposes, or to intact, living subjects. Suitable subjects include mammals of research, agricultural, or economic interest, including rodents, lagomorphs, canines, felines, swine, bovines, and non-human primates. Subjects can include human subjects of any age, sex, or physical condition. Subjects of particular interest include human subjects diagnosed as having, or at risk of developing, a disease condition or medical condition that is, at least in part, affected by the activity of a steroid or steroid receptor. Examples include but are not limited to conditions affected by androgen or the androgen receptor (AR), progesterone or the progesterone receptor (PR), estrogen or the estrogen receptor (ER) and the like. Disease or medical conditions that may be treated with the disclosed methods or compounds include, but are not limited to, carcinomas such as prostate cancer, liver cancer, bladder cancer, cervical cancer, lung cancer and breast cancer, and other cancers which involve the androgen receptor activation pathway, neurological and neuromuscular disorders such as but not limited to Kennedy Disease, skin disorders such as acne, which is caused by androgen-induced AR activation of sebaceous glands, hair disorders such as hirsutism and androgenetic alopecia or “male pattern baldness”, where hair loss is caused by the androgen receptors in follicles and adjacent cells, and wound healing or treatment of inflammation.

Two isoforms (the full-length AR-B and the N-terminus truncated AR-A) of the androgen receptor are expressed in immunologically detectable forms in many fetal and adult human tissues (Wilson and McPhaul (1996)). High AR levels are found in both male and female fetal reproductive tissues, and in varying levels in non-genital fetal tissues. High AR levels are also found in adult reproductive tissues (prostate, endometrium, ovary, uterus, fallopian tube, testis, seminal vesicle, myometrium, and ejaculatory duct), and lower levels in adult breast, colon, lung and adrenal gland tissue. The AR pathway is especially important in the development and proper function of the male reproductive organs as well as non-reproductive organs (including muscle, hair follicles, and the brain). It is involved in the pathology of several diseases or conditions, including prostate cancer, male infertility, and Kennedy's disease.

Prostate cancer is the most common malignancy in American men in terms of incidence and prevalence. It is the most frequently diagnosed neoplasm in the United States and the second leading cause of cancer-related death for American men (Boring et al., 1992). Prostate cancer strikes more than 180,000 men each year, about the same number as cases of breast cancer in women. It caused 31,900 deaths among American men in 1999, second only to lung cancer. The increase in incidence of prostate cancer each year correlates with the aging of American male population.

Prostate cancer cell growth relies upon androgen-induced activation of the androgen receptor (AR). Most prostate cancers are dependent on androgen when first diagnosed (Heinlein and Chang 2004), and thus can be treated with anti-androgens. One effective treatment for metastatic prostate cancer is androgen blockage therapy, which employs either surgical or chemical castration, combined with anti-androgen treatment, to suppress the biological action of androgens (Crawford et al., 1989). However, the median duration of tumor response to steroid depletion is only 18-36 months, and the cancer almost always relapses and becomes androgen non-responsive. In such cases, patients face less desirable therapies, such as chemotherapy. In some cases, alterations of anti-androgens delay the progression of recurrent prostate tumors (Dupont et al, 1993; Taplin et al., 1999), indicating that a prostate tumor which relapses on a specific anti-androgen therapy may respond to a different anti-androgen.

Kennedy Disease—also known as Kennedy's Disease, spinal and bulbar muscular atrophy or spinobulbar muscular atrophy (“SBMA”), or Kennedy's Syndrome) (see, for example, Paul E. Barkhaus (2003), “Kennedy Disease”, electronic publication available on-line at http://www.emedicine.com/neuro/topic421.htm, accessed 15 Apr. 2004—is a rare, X-linked recessive genetic neuromuscular disease that is estimated to affect 1 in 40,000 individuals worldwide. Kennedy disease is believed to be caused by an androgen receptor mutation consisting of an abnormally long polyglutamine expansion in the N-terminus region of the AR gene. It is progressive, and currently incurable and non-treatable. Both the spinal and bulbar neurons are affected, causing muscle weakness and atrophy throughout the body, most noticeably in the extremities and in the face and throat. Kennedy Disease causes speech and swallowing difficulties, major muscle cramps, as well as other symptoms. It is an adult-onset disease with symptoms usually appearing between the ages of 30 and 50, although earlier onsets have been recorded. Only males with this inherited gene develop the full phenotype of the disease, whereas females heterozygous for the gene are generally asymptomatic carriers. In some cases, females who are heterozygous for Kennedy Disease show subclinical phenotypic expression. Life expectancy is generally not affected.

Experimental transfection of cells with a mutated AR having expanded polyglutamine repeats (for example, with the plasmids p6RARQ49 or p6RARQ77) has been shown to be associated with a decreased transactivational function and, in some cases, intranuclear inclusions of misfolded AR proteins (Chamberlain et al. (1994) Nucleic Acid Res., 22: 3181-3186, which is incorporated by reference in its entirety herein). This intranuclear accumulation of abnormal AR is cytotoxic, triggering neuronal cell death, consistent with the in vivo pathology of Kennedy disease.

Inducing Degradation of a Steroid Receptor

The present invention includes methods of inhibiting or reducing steroid-dependent gene activation including administering a compound capable of inducing degradation of a steroid or steroid receptor in cooperation with or together with a second compound capable of inhibiting or reducing gene activation by the steroid receptor, preferably acting at a different point or step along the steroid receptor gene activation pathway. The combination of compounds may provided synergistic, additive or increased inhibition when used in combination. The second compound may inhibit the conversion of a steroid to its more active form, interfere with or inhibit binding between the steroid and steroid receptor, interfere with or inhibit binding between the steroid receptor and corresponding response element, interfere with interactions between a steroid receptor and a cofactor or a steroid receptor associated protein (such as one selected from the ARA family), reduce or prevent nuclear transfer of a steroid receptor, reduce or suppress transcription of the steroid receptor or interfere or suppress translation of the steroid receptor.

When the methods of the present invention are utilized to reduce or inhibit androgen-dependent gene activation, preferably at least one of the administered compounds is capable of inducing degradation of the androgen receptor. Compounds capable of inducing degradation of the androgen receptor include a variety of curcumin derivatives and analogues, such as but not limited to ASCJ-9 and ASCJ-15. Derivatives and analogues encompassed by the present invention include curcumin compounds having altered, reduced or added structural features that may result in degradation of the steroid or androgen receptor.

ASCJ-9, or Dimethylcurcumin, is 5-hydroxy-1,7-Bis-(3,4-dimethoxy-phenyl)-1,4,6-heptatrien-3-one and has the following structure:

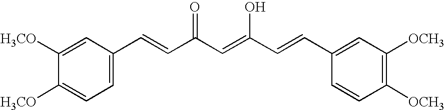

ASCJ-15 is 7-(4-Hydroxy-3-methoxy-phenyl)-4-[3-(4-hydroxy-3-methoxy-phenyl)-acryloyl]-5-oxo-hept-6-enoic acid ethyl ester and has the following structure:

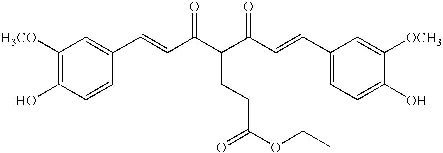

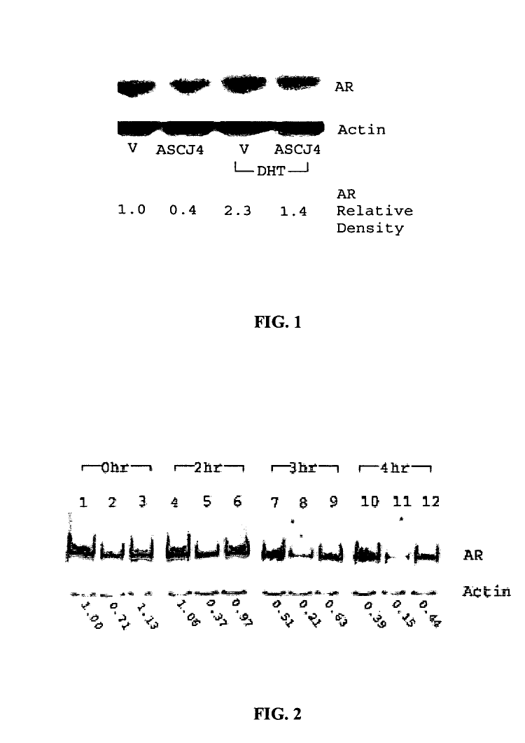

FIG. 1 and FIG. 2 depict the degradation of the androgen receptor via administration of the curcumin derivative ASCJ-15. Compounds ASCJ-9 and ASCJ-15 have similar degradation capabilities. Other curcumin derivative and analogue compounds that may be of particular use are those described in U.S. Pat. No. 6,790,979, which is herein incorporated by reference in its entirety.

One or more mechanisms of steroid receptor degradation may be utilized or induced by the methods or compounds of the present invention. Preferably administration of the compound results in the specific degradation of a targeted steroid receptor, without substantially altering the levels or activity of other, non-targeted steroid receptors.

It may be desirable to induce degradation of a targeted steroid receptor to a different degree in a targeted tissue or cell type. For example, as different tissues or cell types are responsive to a given steroid to different degrees, it may be desirable to induce degradation of the appropriate steroid receptor in one tissue or cell type but not another, or it may be desirable to lower the targeted steroid receptor to different “thresholds” or levels according to the tissue or cell type. In some embodiments, the method may include administering the compound in a quantity sufficient to degrade the targeted steroid receptor in a given tissue or cell type, thus lowering the effect of the steroid receptor to a desired level (for example, where the steroid receptor is the androgen receptor, the desired androgen receptor level may be a level that is substantially non-responsive to circulating androgen).

The methods of the present invention may make use of any one or more suitable mechanisms to induce the degradation of a steroid receptor. These mechanisms include, but are not limited to, interfering with translocation of the steroid receptor into the nucleus or retaining the steroid receptor in the cytoplasm of a cell, exposing a motif within the steroid receptor able to induce protease activity, increasing activity of a protease capable of degrading the steroid receptor, inhibiting the stabilization of a steroid receptor, reducing the solubility of the steroid receptor, activating a pathway able to degrade the steroid receptor, increasing ubiquination of the steroid receptor, increasing phosphorylation of the steroid receptor by an appropriate kinase (for example, in the case of the androgen receptor, by activating Akt kinase, which in at least some cases phosphorylates the androgen receptor, leading to the receptor's ubiquination and subsequent degradation by the proteosome; see, for example, Heinlein and Chang (2004) and Lin et al. (2002) EMBO J, 21:4037-4048, which are herein incorporated by reference in their entirety), inducing apoptosis, or reducing an interaction between a steroid receptor and a cofactor such as ARA able to stabilize the steroid receptor.

In some embodiments, the method of the present invention includes inducing degradation of a steroid receptor by interfering with translocation of the steroid receptor into the nucleus. For example, androgen receptors, like many other steroid receptors are translocated from the cytoplasm to the nucleus where they regulate genes using a zinc-finger motif. When blocking translocation or when retaining the androgen receptor within the cytoplasm, the androgen receptor undergoes proteolysis, and is thus unable to affect the nuclear DNA or regulated gene(s).

In other embodiments, methods and compositions of the present invention induce degradation of a steroid receptor by exposing within the steroid receptor a site or motif that is able to induce proteolysis in the presence of a protease. Such exposure may occur by inducing a conformational change of a domain within the steroid receptor, such as by binding a compound to the steroid receptor or by phosphorylating a domain of the steroid receptor. It is believed that the androgen receptor can be degraded via a ubiquitin-dependent proteosome pathway, or alternatively, by an independent caspase-3-dependent pathway (Lee and Chang (2003)). Evidence supporting the ubiquitin-dependent proteosome pathway includes the existence of a highly conserved PEST (proline-, glutamate-, serine-, and threonine-rich) sequence, thought to target proteins for ubiquitination, in the AR hinge region; and the finding that proteosome inhibition results in elevated AR levels (Lin et al. (2002) EMBO J., 21: 4037-4048). AR is a phosphoprotein and can be phosphorylated by Akt. Constitutive expression of Akt enhances AR ubiquitination and markedly reduces AR levels in the presence or absence of the androgen dihydrotestosterone (DHT); the effect of cAkt can be blocked by the proteosome inhibitor MG132 (Lin et al. (2002)). Additionally, Akt is believed to phosphorylate Mdm2, a ubiquitin ligase (E3 ligase); Mdm2 forms a complex with Akt and AR, serves as the E3 ligase for AR, and promotes AR ubiquitination and subsequent degradation by the proteosome.

The caspase-3-dependent pathway (Lee and Chang (2003) is believed to be independent of the ubiquitin-proteosome pathway. Expression of the tumor suppressor, phosphatase and tensin homologue (PTEN), interfered with AR translocation into the nucleus of LNCaP cells and promoted AR protein degradation. PTEN may interact with the AR DNA-binding domain, leading to AR retention in the cytoplasm and AR degradation.

In another embodiment, the method of the present invention includes inducing degradation of a steroid receptor by preventing or reducing stabilization of the steroid receptor. Preventing or decreasing stabilization may occur by inhibiting interactions between two or more steroid receptors or by preventing or decreasing the interaction between a steroid receptor and a cofactor. In one example, the androgen receptor is believed to dimerize with a second copy of itself forming a homodimer, which increases the stability of the receptor. Dimerization may occur via interaction between the amino terminals of the receptors. Preventing dimerization, for example, by administering a compound able to reduce or eliminate dimerization by binding at or near the amino terminal domain, may induce the degradation of the androgen receptor and thus inhibit the androgen receptor activation pathway. In another embodiment, the interaction between a steroid receptor and a cofactor may be disrupted, thereby inducing degradation of the steroid receptor. For example, heat shock protein (HSP) is a cofactor that binds to and stabilizes the androgen receptor (as well as serving as chaperone to other proteins). HSP is believed to bind the amino terminal domain of the androgen receptor. Specifically reducing cofactor binding and stabilization, for example, by administering a compound able to specifically block the binding between HSP and the amino terminal domain (without affecting HSP's binding to other proteins it chaperones), may induce the degradation of the androgen receptor and thus inhibit the androgen receptor-activated pathway. In these non-limiting examples, the compound may disrupt dimerization or cofactor binding by interacting at any location or locations on the steroid receptor.

In another embodiment, the method of the present invention may include administering a compound capable of interfering with or inhibiting binding between a steroid receptor and a coregulator or cofactor. Accessory factors such as androgen receptor associated (ARA) proteins bind to steroid receptors, such as the androgen receptor, and may provide stability and may determine the specificity of gene activation by the steroid receptor. That is, the binding of ARA proteins may assist in the determination of which genes are affected by the steroid receptor. Multiple androgen receptor associated (ARA) proteins have been identified and it is envisioned that additional ARA proteins will be discovered in the future. ARA proteins may bind near the amino terminal or the carboxyl terminal regions of the androgen receptor (AR). Examples of ARA proteins that may be targeted by the present invention include, but are not limited to ARA24, ARA54, ARA70, ARA267-α, ARA derivatives, ARA analogues and the like.

In another embodiment, the method of the present invention induces degradation of a steroid receptor by destabilization of a domain of the steroid receptor. For example, some studies suggest that the AF-2 domain of the androgen receptor stabilizes the receptor's overall structure, allowing the amino terminal domain to interact with coregulators. A compound able to destabilize the AF-2 domain, for example, by interacting or binding with the AF-2 domain or with a domain that itself interacts with the AF-2 domain, may reduce the stabilization of the amino terminal domain, reduce the interaction with coregulators, and increase the rate of degradation of the androgen receptor.

In another embodiment, the method of the present invention induces degradation of a steroid receptor by activating a pathway able to degrade the steroid receptor. For example, the caspase-3 pathway has been suggested to induce degradation of the androgen receptor. Activation of the caspase-3 pathway may occur by the presence of PTEN such as by an interaction between the DBD and the PTEN phosphatase domain. Therefore a compound able to induce the caspase-3 pathway may induce the degradation of the androgen receptor. In another example, Akt kinase, which phosphorylates the androgen receptor, may be activated, leading to ubiquination and subsequent degradation of the androgen receptor by the proteosome (Heinlein and Chang 2004).

Interfering With the Conversion of a Steroid to its Active Form

In another embodiment the present invention includes administering a compound capable of inducing degradation of a steroid receptor in combination with a compound capable of inhibiting or suppressing conversion of a steroid to its active or alternative form. For example, testosterone is converted to dihydrotestosterone (DHT) by the enzyme 5-alpha-reductase. Administration of a compound capable of inhibiting or suppressing enzyme 5-alpha-reductase (5AR) activity would decrease the presence of DHT and therefore suppress or reduced steroid dependent gene activation. Examples of suitable inhibitors include dutasteride, finasteride and the like. Thus, administering a compound capable of inhibiting the conversion from testosterone to DHT in combination with a curcumin derivative or analogue may provide an efficient anti-androgenic effect and may provide an effective treatment to a variety of androgen associated disorders.

Interfering with Binding Between A Steroid and Steroid Receptor

The present invention also includes administering a compound capable of degrading a steroid receptor in combination or together with a compound capable of inhibiting or reducing steroid-dependent gene activation by interfering with or inhibiting the binding between a steroid and a steroid receptor.

The two compounds may be administered in a single composition such as a pharmaceutical composition or cosmetic composition or may be administered in separate compositions. The two compounds are provided for treatment of the same medical condition.

Compounds capable of interfering with binding between a steroid and a steroid receptor may affect the steroid, the steroid receptor or both. For example, the compound may attach entirely or in part to the steroid binding site of the steroid receptor thereby preventing binding between the steroid and steroid receptor. Alternatively, the compound may bind outside or partially outside of the steroid binding site of the steroid receptor. Attachment or binding of the compound to the steroid receptor may result in a conformational change of the steroid receptor thereby decreasing the ability for the steroid and steroid receptor to bind one another. Examples of compounds able to interfere with binding between a steroid and a steroid receptor include but are not limited to bicalutamide, flutamide and functional derivatives or analogues thereof.

Interfering with binding between a steroid and steroid receptor is a therapy currently used for a variety of steroid associated disorders or medical conditions. For example, flutamide and bicalutamide (Casodex) developed by pharmaceutical companies are non-steroid antiandrogens that have been used to treat prostate cancer. However, tumors frequently relapse and become hormone refactory. The present invention includes the administration of one or more of the above compounds in combination or together with a compound capable of inducing degradation of the androgen receptor, such as but not limited to a curcumin derivative or analogue. The present invention may provide greater inhibition of androgen induced gene activation and may provide a more effective treatment than flutamide or bicalutamide alone.

Interfering with A Steroid Receptor Binding to A Steroid Response Element

The present invention also includes the administration of a compound capable of inducing degradation of the androgen receptor and a compound capable of reducing, suppressing or interfering with binding between a steroid receptor and a steroid response element (also referred to as a steroid receptor response element or corresponding response element).

The steroid response element (SRE) is an endogenous DNA sequence typically positioned within the promoter region of the target gene and is the DNA binding target of the steroid receptor. When the steroid receptor, usually having a zinc finger motif, binds to the corresponding response element, the steroid receptor initiates transcription. Nonlimiting examples of steroid response elements that may be targeted with one or more methods and compositions of the present invention include the androgen response element (ARE), progesterone response element (PRE), the estrogen response element (ERE), the glucocortocoid response element (GRE) and the like.

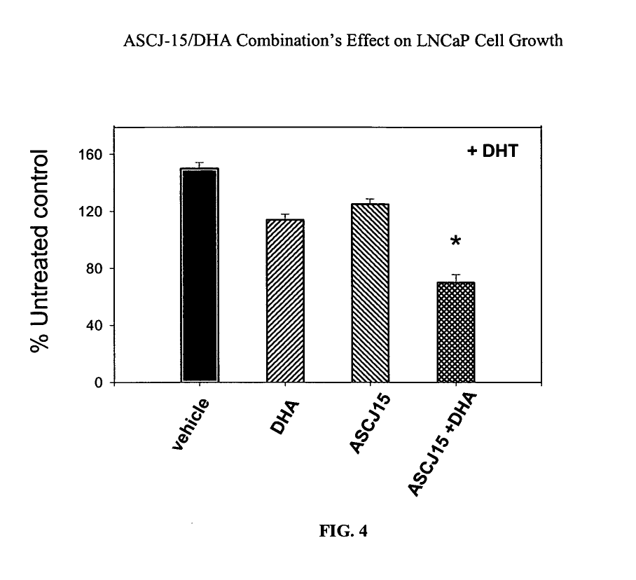

Compounds of the present invention that may interfere or inhibit binding between a steroid receptor and a corresponding response element include but are not limited to docosahexaenoic acid (DHA), functional derivatives of DHA and the like. The effect of DHA on androgen-mediated gene activation has previously been studied. Chung et. al., Effects of docosahexaenoic acid and eicosapentaenoic acid on androgen-mediated cell growth and gene expression in LNCaP prostate cancer cells. Carcinogenesis 22: 1201-1206, 2001. The present invention demonstrates the ability of DHA to suppress androgen receptor binding to the androgen response element (ARE) and its combination with a compound capable of inducing degradation of the androgen receptor. Using the androgen receptor (AR) transactivation methods disclosed in Example 1 and with results further disclosed in Example 3 and FIG. 4, when DHA was used in combination with ASCJ-15, a curcumin derivative, a synergistic effect was found in suppressing prostate cancer cell growth in vitro.

Suppressing Nuclear Transfer of a Steroid Receptor

The present invention also includes methods and compositions capable of inhibiting or reducing steroid-dependent gene activation utilizing a compound capable of inducing degradation of a steroid receptor in combination with or together with a compound capable of inhibiting or reducing, at least in part, nuclear transfer of a steroid receptor.

Steroid receptors are translocated from the cytoplasm to the nucleus where the steroid receptor is capable of binding to the steroid response element (SRE). Therefore compounds and methods of the present invention capable of reducing or inhibiting nuclear transfer of the steroid receptor would allow reduction or inhibition of steroid-dependent gene activation. Thus, when administered in combination with a compound capable of degrading the steroid receptor such as a curcumin derivative or analogue, improved inhibition may be obtained.

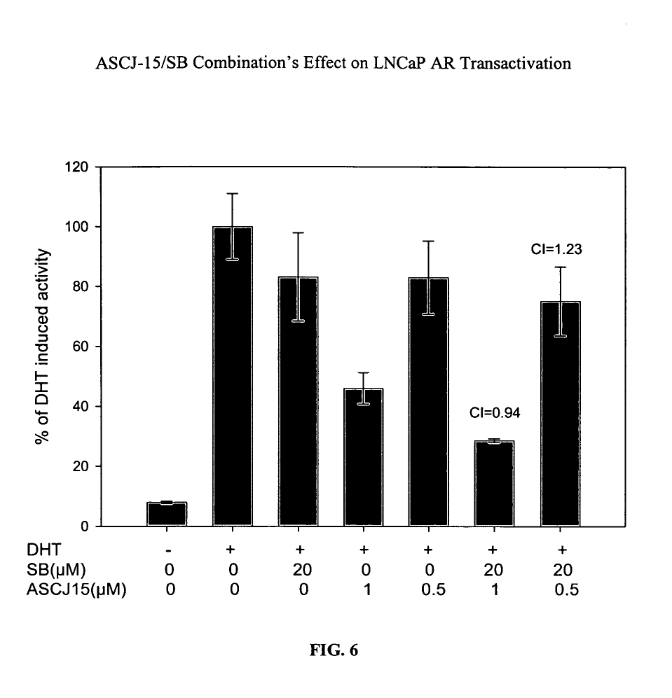

Examples of compounds capable of reducing or inhibiting, at least in part, nuclear transfer of a steroid receptor may act on the steroid-receptor complex when located in the cytoplasm of a cell. Compounds may target or induce conformational changes in the steroid receptor, may prevent dimerization of the steroid receptor, may interfere with the binding of cofactors to the steroid receptor and the like. Specific examples of compounds encompassed by the present invention include but are not limited to silibinin (SB) or functional derivatives thereof. Silibinin has be previously demonstrated to prevent nuclear transfer of the androgen receptor. Zhu et. al., Silymarin inhibits function of the androgen receptor by reducing nuclear localization of the receptor in the human prostate cancer cell line LNCaP, Carcinogenesis 22: 1399-1403, 2001. Example 5 and FIG. 6 demonstrate Silibinin (SB) and ASCJ-15, a curcumin derirvative, are capable of acting in an additive fashion in the suppression of androgen-induced gene activation. Silibinin (SB) and ASCJ-15 were also shown to suppress prostate cancer cell growth in an additive fashion indicating SB and ASCJ-15 may have significant therapeutic utility in the treatment of steroid associated medical conditions.

Reducing or Suppressing Transcription or Translation of A Steroid Receptor

The present invention also includes methods and compositions capable of inhibiting or reducing steroid-dependent gene activation including administering a compound capapble of inducing degradation of a steroid receptor and administering a compound capable of reducing or suppressing transcription of a steroid receptor mRNA.

As is commonly known in the molecular biology arts, DNA or cDNA is transcribed into mRNA, which is translated or expressed as protein. Thus, expression and proper display of a steroid receptor requires the steroid receptor gene sequence be transcribed properly into the corresponding mRNA. Suppressing or reducing transcription of the steroid gene sequence may decrease the expression of the corresponding steroid receptor protein and would allow partial inhibition of steroid-dependent gene activation.

Suppression of steroid receptor transcription may utilize a variety of suppression techniques known in the molecular biology arts. Quercetin (QU) has previously been shown to inhibit gene activation by the androgen receptor in LNCaP cells. Xing et. al., Quercetin inhibits the expression and function of the androgen receptor in LNCaP prostate cancer cells. Carcinogenesis 22: 409-414, 2001. Techniques encompassed by the present invention include but are not limited to suppressing or reducing promoter activity, activation or stimulation of a transcription factor able to suppress transcription of the steroid receptor, interfering with polymerase activity, utilizing small interfering RNA (siRNA), utilization of anti-sense technology and the like. Examples of compounds capable of suppressing androgen receptor transcription include but are not limited to quercetin (QU), vitamin E succinate (VES) and functional derivatives thereof.

In addition methods and compositions of the present invention include those that reduce or suppress translation of the steroid receptor. The disclosed compounds and methods may utilize any known method of suppressing translation of a steroid receptor such as but not limited to effecting regulatory compounds or sequences involved in the expression of a steroid receptor or utilization of anti-sense technology.

II. Compositions Capable of Inhibiting or Reducing Steroid-dependent Gene Activation

The present invention also includes compositions capable of inhibiting or reducing steroid-dependent gene activation utilizing at least two compounds, each capable of inhibiting or reducing steroid-dependent gene activation. In some embodiments one compound is capable of inducing degradation of a steroid receptor and the second compound is capable of inhibiting or reducing steroid-dependent gene activation by acting at a different point or step along the steroid receptor pathway. Suppression or inhibition of steroid-dependent gene activation may be increased by administering the at least two compounds in combination or together in comparison to administration only a single compound. The second compound may inhibit one or more steps along the steroid receptor pathway such as but not limited to: inhibiting or reducing the binding between a steroid and the corresponding steroid receptor, inhibiting or reducing the binding between a steroid receptor and a steroid response element, interfering with an interaction between a steroid receptor and a cofactor or a steroid receptor associated protein (such as ARA), inhibiting or reducing nuclear transfer of a steroid receptor, reducing or preventing transcription of the steroid receptor, reducing or inhibiting translation of a steroid receptor, and the like.

When inhibiting or reducing androgen-dependent gene activation, preferably the composition includes at least one compound capable of inducing degradation of the androgen receptor and more preferably the compound is a curcumin derivative, such as ASCJ-9, ASCJ-15 and the like. Nonlimiting examples of compounds capable of inhibiting the conversion of testosterone to dihydrotestosterone (DHT) include 5-alpha-reductase inhibitors such as dutasteride, finasteride and functional derivatives and analogues and the like. Non-limiting examples of compounds capable of inhibiting binding between and androgen and the androgen receptor include but are not limited to bicalutamide, hyroxyflutamide and functional derivatives and analogues and the like. Non-limiting examples of compounds capable of inhibiting binding between an androgen receptor and the androgen response element (ARE) include docosahexaenoic acid (DHA) and functional derivatives and analogues and the like. Non-limiting examples of compounds capable of inhibiting nuclear transfer of an androgen receptor include silibinin (SB) and functional derivatives and analogues and the like. Non-liming examples of compounds capable of inhibiting transcription of the androgen receptor include vitamin E succinate (VES), quercetin (QU) and functional derivatives and analogues and the like.

The compositions of the present invention may include pharmaceutical compositions or cosmetic compositions capable of treating medical conditions, at least in part, associated with steroid-dependent gene activation. Steroid receptors of particular interest may include but are not limited to androgen receptors (AR), progesterone receptors (PR), estrogen receptors (ER), glucocorticoid receptors (GR), peroxisome proliferator-activated receptor (PPAR), retinoid X receptors (RXR), and orphan steroid hormone receptors. Medical conditions may include but are not limited cancers such as but not limited to prostate cancer, liver cancer, bladder cancer, cervical cancer, lung cancer and breast cancer, neurological and neuromuscular disorders such as Kennedy Disease, skin disorders such as acne, hair disorders such as androgenetic alopecia or “male pattern baldness”, where hair loss is caused by androgen activity on the androgen receptors in follicles and adjacent cells, hirsutism and wound healing and inflammation.

The compositions of the present invention may include two or more active compounds or effective analogues, variations or derivatives thereof in a suitable carrier. Any suitable active compounds may be of use, if they are capable of inhibiting or reducing steroid-dependent gene activation. When targeting androgen associated medical conditions, preferably at least one compound is effective at inducing degradation of the androgen receptor at physiologically acceptable levels, such as at levels that do not cause substantial undesirable side effects or toxicity. The active compound can optionally include one or more elements that provide additional benefits, such as improved stability, solubility, or delivery specificity. Such elements can include peptides, polypeptides, proteins, carbohydrates, nucleic acids, lipophilic moieties, hydrophilic moieties, particulates, matrices, or combinations thereof. For example, the active compound can be linked, covalently or non-covalently, to a hydrophilic moiety (such as a phosphate or sulphate group or a carbohydrate or a chelating molecule) to improve solubility in aqueous buffers or bodily fluids. In another example, the active compound can be linked, covalently or non-covalently, to a peptide or other moiety that protects the active compound from premature degradation, or to an antibody or other specific binding agent that specifically targets a desired tissue or cell type and thus improves delivery of the active compound to that specific tissue or cell type. In another example, the active compound can be encapsulated or embedded in a liposome, a particulate, a matrix, a gel, a polymer, or the like, to improve stability or to enhance delivery.

Suitable carriers of use in the compositions of the invention include diluents, excipients, or carrier materials, selected according to the intended form of administration and consistent with conventional pharmaceutical or cosmetic practice. Examples of suitable carriers include, but are not limited to, water, physiological saline, phosphate-buffered saline, a physiologically compatible buffer, saline buffered with a physiologically compatible salt, a water-in-oil emulsion, and an oil-in-water emulsion, an alcohol, dimethylsulfoxide, dextrose, mannitol, lactose, glycerin, propylene glycol, polyethylene glycol, polyvinylpyrrolidone, lecithin, albumin, sodium glutamate, cysteine hydrochloride, and the like, and mixtures thereof. Suitable carriers can also include appropriate pharmaceutically acceptable antioxidants or reducing agents, preservatives, suspending agents, solubilizers, stabilizers, chelating agents, complexing agents, viscomodulators, disintegrating agents, binders, flavoring agents, coloring agents, odorants, opacifiers, wetting agents, pH buffering agents, and mixtures thereof, as is consistent with conventional pharmaceutical practice (“Remington: The Science and Practice of Pharmacy”, 20th edition, Gennaro (ed.) and Gennaro, Lippincott, Williams & Wilkins, 2000).

For use in isolated cells, such as in cells grown in culture and used in bioassays, compositions of the present invention can be formulated and provided as is convenient. In non-limiting examples, compositions may be formulated as dissolvable solids, solutions, suspension, liposome preparations, and the like, and provided to the cells by manual or automated delivery (such as by pipette, syringe, pump, auto-injector, and the like).

For use in a living, whole organism, such as in a human subject, compositions of the present invention can be formulated and provided in any formulation suitable to the intended form of administration and consistent with conventional pharmaceutical practice (“Remington: The Science and Practice of Pharmacy”, 20^{th} edition, Gennaro (ed.) and Gennaro, Lippincott, Williams & Wilkins, 2000). Examples of suitable formulations include tablets, capsules, syrups, elixirs, ointments, creams, lotions, sprays, aerosols, inhalants, solids, powders, particulates, gels, suppositories, concentrates, emulsions, liposomes, microspheres, dissolvable matrices, sterile solutions, suspensions, or injectables, and the like. Injectables can be prepared in conventional forms either as liquid solutions or suspensions, as concentrates or solid forms suitable for solution or suspension in liquid prior to injection, or as emulsions.

For use in a living, whole organism, such as in a human subject, and depending on the specific conditions being treated, pharmaceutical compositions of the present invention can be formulated and administered systemically or locally. Techniques for formulation and administration can be found in “Remington: The Science and Practice of Pharmacy” (20^{th} edition, Gennaro (ed.) and Gennaro, Lippincott, Williams & Wilkins, 2000). Suitable routes of administration can include oral, intestinal, parenteral, transmucosal, transdermal, intramuscular, subcutaneous, transdermal, rectal, intramedullary, intrathecal, intravenous, intraventricular, intraatrial, intraaortal, intraarterial, or intraperitoneal administration. The pharmaceutical compositions of the present invention can be administered to the subject by a medical device, such as, but not limited to, implantable devices, biodegradable implants, patches, and pumps. Where such a device is used, the compositions may be formulated to include a dissolvable or nondissolvable matrix or medium (for example, a coating, membrane, film, impregnated matrix, polymer, sponge, gel, or porous layer on or within the medical device) to permit the release of the active compound or compounds over a specified period of time.

III. Methods of Preventing or Treating a Steroid Modulated Medical Condition

The present invention also includes methods of treating or preventing a steroid modulated condition. Examples of conditions that may be treated with the disclosed methods include but are not limited to cancer, such as prostate cancer, liver cancer, bladder cancer, cervical cancer, lung cancer and breast cancer, and other cancers which involve the androgen receptor activation pathway, neurological and neuromuscular disorders such as but not limited to Kennedy Disease, skin disorders such as acne, which is caused by androgen-induced AR activation of sebaceous glands, hair disorders such as androgenetic alopecia or “male pattern baldness”, where hair loss is caused by the androgen acting on androgen receptors in follicles and adjacent cells, hirsutism, and wound healing or treatment of inflammation.

The methods of preventing or treating a steroid modulated medical condition include providing an individual such as a human, suspected of suffering from or at risk of developing a steroid modulated medical condition and administering a therapeutically effective amount of a pharmaceutical including at least two compounds, optionally in a suitable carrier, each capable of inhibiting or suppressing steroid-dependent gene activation. The pharmaceutical may be any previously disclosed in the above provided methods or compositions. Preferably at least two of the at least two compounds act at different stages or points of the steroid receptor gene activation pathway. When the steroid modulated medical condition is an androgen-modulated medical condition, preferably at least one of the compounds is capable of inducing degradation of the androgen receptor and more preferably the compound is a curcumin derivative or analogue such as but not limited to ASCJ-9 or ASCJ-15.

The present invention also includes methods of treating a wound or inflammation including providing an individual having a wound site or inflammatory region and topically administering a composition to the site or region. The composition may include at least two compounds, each capable of inhibiting steroid-dependent gene activation.

The compounds should act at different points of the steroid receptor gene activation pathway as provided above.

The present invention also includes a method of inhibiting or reducing steroid-dependent gene activation in a human including providing at least one of the compositions provided above, encapsulating the composition in a liposome and administering the encapsulated composition to a human.

### EXAMPLES

### Example 1

### Detecting Changes in Steroid-induced Gene Activation using an Androgen Receptor Transactivation Assay, an MTT Cell Proliferation Assay and Western Blot Analysys

The provided examples provide a representative study of the effects of administering of a combination of compounds, at least two of which are capable of affecting at different steps along a steroid-dependent gene activation pathway. The provide examples include an androgen receptor (AR) transactivation assay to study the effect that the various combinations of compounds have on the activity of a wild-type or a mutant androgen receptor found in prostate cancer and an MTT cell proliferation assay to test the ability of the combination of compounds to suppress prostate tumor cell growth. Preferred assays utilize human prostate carcinoma cells, LNCaP cells, which are accepted by those skilled in the art as expressing a clinically relevant mutant androgen receptor (AR) that is responsive to androgen. In the provided examples, dihydroxytestosterone (DHT), the endogenous hormone metabolite of testosterone, is administered to LNCaP cells to induce activity of the androgen receptor (AR) in the androgen receptor (AR) transactivation assay and to stimulate carcinoma cell growth in the MTT cell proliferation assay. When provided in combination the disclosed assays provide guidance for the development of potential treatments of various steroid-dependent medical conditions.

Androgen Receptor (AR) Transactivation Assay

The androgen receptor (AR) transactivation assay was chosen as the primary tool to assess the effect of ASCJ compounds, which are curcumin derivates, in combination with docosahexaenoic acid (DHA), silibinin (SB), quercetin (QU) and hydroxyflutamide (HF). The androgen receptor (AR) transactivation assay measures the end point of the androgen receptor (AR) pathway-target gene expression, via detection of a luciferase reporter gene. Methods of detecting anti-androgen activity have previously been shown. Ohtsu et. al. Synthesis and Anti-androgen Activity of New Diarylheptanoids. Bioorg Med Chem 11: 5083-90, 2003 and Curcumin Analogues as Novel Androgen Receptor Antagonists with Potential as Anti-prostate Cancer Agents. J Med Chem 45, 5037-5042, 2002. Luciferase reporting systems have been previously demonstrated. Sherf, B. A. et al. (1996) Dual-Luciferase reporter assay: an advanced co-reporter technology integrating firefly Renilla luciferase assays. Promega Notes 57, 2. Data derived from the assay should reflect the magnitude of cooperation among tested compounds in blocking, suppressing or inhibiting the AR activation pathway.

The androgen receptor (AR) transactivation assay includes a cell population or cell line, such as LNCaP cells, transfected with a plasmid including an androgen response element integrated within or nearby a promoter and a luciferase reporting system positioned downstream of the promoter. Changes in detection or measurement of the quantity of luciferase in the presence of test compounds correlate to modulation, suppression or inhibition of gene expression by the test compounds.

More specifically, human prostate cancer cells, LNCaP (ATCC, Manassas, Va.), were maintained in RPMI-1640 medium supplemented with 10% heat-inactivated fetal bovine serum (FBS), L-glutamine (300 mg/L), and penicillin-streptomycin (100 units/mL penicillin G sodium, 100 μg/mL streptomycin sulfate). For performing assays, cells were plated in 24-well tissue culture plates at a concentration of 1×10⁵ cells/well and cultured at 37° C. in an incubator with 5% CO₂. Two days later, the cells were transfected with SuperFect Transfection Reagent (Qiagen) and DNA mixtures consisting of an MMTV-luciferase plasmid containing an androgen responsive element (ARE) in the promoter region (0.5 μg/well), and a pRL Renilla plasmid under the control of the SV40 promoter (5 ng/well). Following transfection, the cells were fed with charcoal/dextran treated FBS containing medium and treated with one or a combination of two experimental compounds optionally with dihydroxytestosterone (DHT), according to the experiment design. Twenty hours after treatment, the cells were lysed and the luciferase activity in the lysate was analyzed using the Dual-Luciferase Reporter Assay System (Promega).

MTT Cell Proliferation Assay

The MTT cell proliferation assay is used in the present invention to detect the ability for various combinations of compounds to modulate, suppress or inhibit carcinoma cell growth. The cells used are the clinically relevant LNCaP cells. Cell growth is stimulated by the addition of dihydroxytestosterone (DHT). Thus, the MTT cell proliferation assay may provide guidance as to which combination of compounds may be useful at inhibiting or suppressing prostate tumor cell growth.

The MTT assay, which relies upon the conversion of colorless substrate to reduced tetarzolium by a mitochondrial dehydrogenase (possess by all viable cells), has been employed to assess the growth of LNCaP cells (Su et al., 1999). Briefly, 1×10³ LNCaP cells/well are plated in 96-well microtest III tissue culture plates (Falcon, N.J.). Two days later, the medium is changed to RPMI containing 10% charcoal/dextran-deprived FBS. The one or more test compounds are added at indicated concentrations with or without 0.1 nM DHT for 5 days. MTT solution (5 mg/ml in PBS) in 1/10 of volume is added to the cells for 2 h at 37° C. The plates are centrifuged (10 min, at 1,000 rpm) and then supernatant from each well is carefully removed, 100 μl of lysis buffer (50% dimethyl formamide, 5% sodium dedecyl sulphate, 0.35 M acetic acid and 50 mM HCl) is added to each well to lyse the tetrazolium in each well. Viable cell numbers are measured relative to the quantity of enzyme activity from each well (absorbance read at a wavelength of 595 nm using a Bio-RAD BenchMark microplate reader). Data derived from the MTT assay is also verified by actual cell count and morphology.

Normal and non-prostate tumor cells were also used in proliferation assay. Human endothelial cells were obtained from Clonetics (MD) and maintained in EGM supplemented with human epithelial growth factor, hydrocortisone, gentamycin, amphotericin B, and 2% FBS. To set up the assay, the cells were seeded at a density of 5×10³/well in 96-well tissue culture plates. Twenty-four hours later, the cells were fed with the same medium supplemented with 2% charcoal/dextran-deprived FBS. TCHM extracts will be added at various concentrations (0.1-10 μg/ml) to the cells for 5 days. The MTT solution was then added and cell proliferation activity measured as described in the previous paragraph. Again, data derived from the MTT assay were verified using actual cell count and cell morphology.

Western Blot Analysis

A Western blotting method was employed to measure expression of the androgen receptor (AR). The Western blotting method has been published previously (Su et al., 1999). Briefly, cells were harvested either in 2×SDS loading buffer or in RIPA lysis buffer plus 10 μg/ml of benzamidine, 10 μg/ml of trypsin inhibitor, and 1 mM of phenylmethylsulfonyl fluoride. Total protein (40 μg/sample) from cell lysate was separated on a SDS-PAGE gel. After separation, the proteins were transferred from the gel to a nitrocellulose membrane following standard procedures. The membrane was incubated with 10% non-fat milk in phosphate-buffered saline supplemented with 0.1% Tween-20 (PBST) overnight. Primary antibodies specific for human androgen receptor (AR) (BD-PharMingen) were added to the membrane at 4° C. overnight or at room temperature for 2 hours. The membrane was rinsed with PBST buffer three times, 10 min each time, and then added with appropriate horseradish peroxidase-conjugated secondary antibody for 1 hour at room temperature. After rinsed with PBST again, the androgen receptor (AR) protein signal in the membrane was visualized using enhanced chemiluminescence substrates (ECL, Amersham). To assure equal loading among the samples, the membrane was stripped following the manufacture's recommendations and re-incubated with a specific antibody to β-actin (Sigma).

### Example 2

### Inducing Degradation of the Androgen Receptor by Administration of a Curcumin Derivative

Western blot analysis of LNCaP cell lysates was used to depict the reduced presence of the androgen receptor (AR) when LNCaP cells were cultured in the presence of a curcumin derivative, termed an ASCJ compound. A decrease in the presence of the androgen receptor (AR) was found when the ASCJ compound was cultured alone in LNCaP cells, when ASCJ was cocultured with androgen DHT and when ASCJ was cocultured with cyclohexamide, a protein synthesis inhibitor.

Specifically, LNCaP cells were cultivated in 10% CD/RPMI and were treated with ASCJ-15 (1 μM), a curcumin derivative, or a control vehicle in the presence or absence of DHT (2 nM) for 24 hours. Androgen receptor (AR) protein expression from each cell sample was analyzed by western blot as described in Example 1. The experiment was performed four times to ensure data reproducibility. Representative data from one experiment are shown in FIG. 1. The relative androgen receptor (AR) density was derived by normalizing the androgen receptor (AR) signal with that of B-actin and is also provided in FIG. 1.

To demonstrate the decreased presence of the androgen receptor (AR) was due to degradation and not due to regulation of transcription or translation of the androgen receptor (AR), LNCaP cells were exposed to cyclohexamide, a protein synthesis inhibitor. Referring to FIG. 2, LNCaP cells were cultured in 10% CD/RPMI with vehicle control (lanes 1, 4, 7, 10) and treated with ASCJ-15 (1 μM, lanes 2, 5, 8 and 11) or with vitamin E succinate (VES) (10 μM, lanes 3, 6, 9 and 12), for 20 hours. Subsequently, cycloheximide, a protein synthesis inhibitor was added to all cultured cells (15 μg/ml, in all lanes) for the designated times (2, 3 and 4 hr). The expression of the androgen receptor (AR) from each cell samples was analyzed by Western blot analysis as described in Example 1. The data was compared relative to the androgen receptor (AR) density to the 0 hr control sample. Representative data from a single experiment are shown in FIG. 2.

### Example 3

### Inhibiting Steroid-induced Gene Activation by Administering a Compound Capable of Degrading a Steroid Receptor Alone or in Combination with a Compound Capable of Blocking Binding Between the Steroid Receptor and Corresponding Steroid Response Element (SRE)

The effect of a compound capable of degrading an androgen receptor (AR) was administered alone and in combination with a compound capable of blocking or interfering with binding between an androgen receptor and an androgen response element (ARE). The curcumin derivative, ASCJ, was shown in Example 2 as a compound capable of inducing degradation of the androgen receptor (AR). Compound ASCJ-15 was provided alone and in combination with docosahexaenoic acid (DHA), an omega-3 fatty acid capable of blocking or interfering with binding between the androgen receptor (AR) and the androgen response element (ARE). The effect of DHA on androgen-mediated gene activation has previously been studied. Chung et. al., Effects of docosahexaenoic acid and eicosapentaenoic acid on androgen-mediated cell growth and gene expression in LNCaP prostate cancer cells. Carcinogenesis 22: 1201-1206, 2001. In the present example, a combination of compounds, which act at different points of the androgen receptor (AR) pathway were tested in both the androgen receptor (AR) transactivation assay and the MTT cell proliferation assay described in Example 1.

ASCJ-15 was administered at 0, 0.5 and 1.0 μM in the androgen receptor (AR) transactivation assay using LNCaP cells as described in Example 1. The suboptimal dose of 0.5 μM was included to ensure the detection of any modulation of androgen receptor (AR) activity between ASCJ-15 and DHA, whether activity increased or decreased. DHA was administered at the previously reported minimal effective dose of 150 μM.

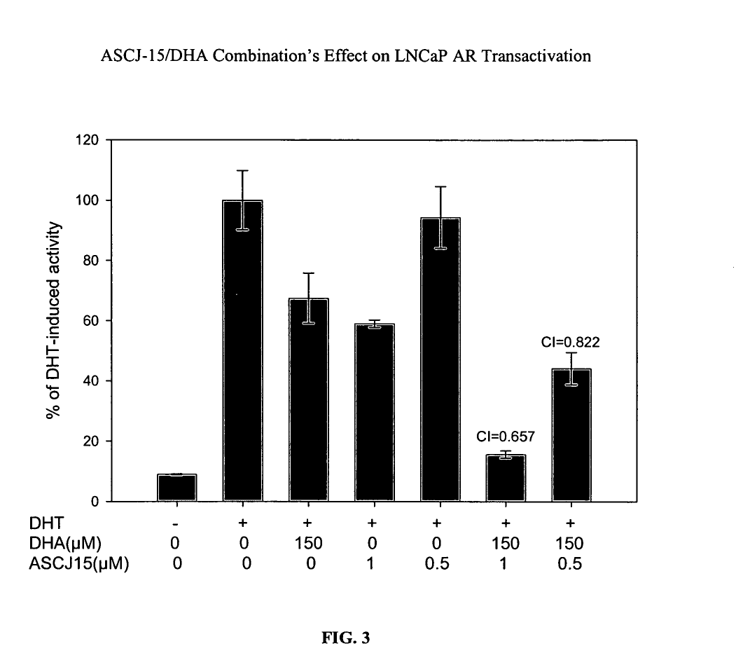

More specifically, the androgen receptor (AR) transactivation assay described in Example 1 above was utilized in transfected LNCaP cells. Cells were grown for 24 hours with 1 nM of DHT in the absence or presence of ASCJ-15 or DHA. FIG. 3 depicts the results of the experiment. DHT incubation raised MMTV-luciferase expression above the basal level by >10 fold. Administration of 150 μM of DHA or 1 μM of ASCJ-15 to the cells suppressed DHT-induced reporter gene expression by 30% or 45%, respectively. The concomitant addition of 150 μM of DHA and 1 μM of ASCJ-15 lowered the induced MMTV luciferase by 84%; an effect that is greater than the addition of the respective effects achieved by each compound alone. Treatment with sub-optimal dose (0.5 μM) of ASCJ-15, together with 150 μM of DHA down-regulated the induced reporter gene expression by a magnitude of 55%; this suppression is significantly greater than that produced by DHA treatment alone (30%). These data suggest that combining DHA and ASCJ-15 could produce a synergistic effect in suppression of AR activity.

ASCJ-15 and DHA were also tested alone and in combination for the efficacy in control of human prostate tumor cell growth. LNCaP cells were used in the MTT assay as described in Example 1. The results are depicted in FIG. 4. DHT significantly stimulated the growth of LNCaP cells by 150%. The addition of 150 μM of DHA to DHT-treated cells attenuated the growth by 30%. Treatment of cells with 1 μM of ASCJ-15 lowered the induced growth by 22%. When ASCJ-15 and DHA were administered in combination, DHT-stimulated growth was suppressed by 64%, which was approximately equal to, or greater than, the sum of the respective effect produced by ASCJ-15 and DHA. These results are consistent with data from the androgen receptor (AR) transactivaton assay, i.e., there is synergistic effect by combining ASCJ compound and DHA in suppressing prostate cancer cell growth.

### Example 4

### Inhibiting Steroid-induced Gene Activation by Administering a Compound Capable of Reducing Binding Between a Steroid and a Corresponding Steroid Receptor in Combination with a Compound Capable of Reducing Binding Between a Steroid Receptor and a Steroid Response Element

The effect of a compound capable of reducing binding between an androgen and an androgen receptor (AR) was administered alone and in combination with a compound capable of blocking or interfering with binding between an androgen receptor and an androgen response element (ARE). Hydroxyflutamide (HF), a metabolite of flutamide, is believed to block binding between androgen and the androgen receptor. Docosahexaenoic acid (DHA), an omega-3 fatty acid, is believed to block or interfere with binding between the androgen receptor (AR) and the androgen response element (ARE). The two compounds were administered alone and in combination to LNCaP cells using the androgen receptor (AR) transactivation assay as described in Example 1.

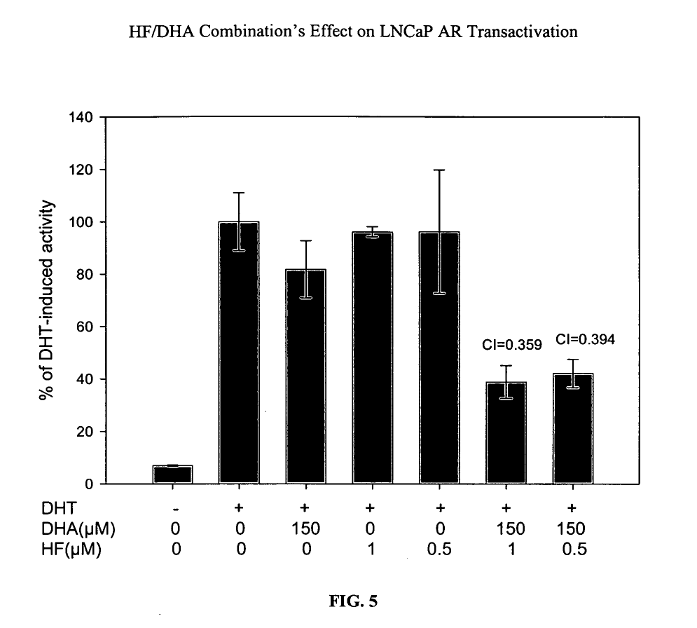

As in Example 3, DHA was used at its minimal effective dose, 150 μM, in the androgen receptor (AR) transactivation assay. HF was provided at 0, 0.5 or 1.0 μM. DHT was provided at 1×10⁻⁹M. The results are depicted in FIG. 5. Both DHA and HF were capable of suppressing DHT-induced androgen activity alone and in combination. A synergistic anti-androgenic effect was observed when DHA and HF were used in combination resulting in greater suppression of DHT-induced activity.

### Example 5

### Inhibiting Steroid-induced Gene Activation by Administering a Compound Capable of Inducing Degradation of a Steroid Receptor in Combination with a Compound Capable of Blocking Nuclear Transfer of a Steroid Receptor

The effect of a compound capable of inducing degradation of an androgen receptor (AR) was administered alone and in combination with a compound capable of blocking or interfering with nuclear transfer of an androgen receptor. ASCJ-15 as demonstrated in Example 2 is capable of degrading the androgen receptor. A purified form of Silibinin (SB), a polyphenolic flavonoid abundant in the widely consumed milk thistle, is believed to interfere with nuclear transfer of an androgen receptor. Each were administered alone and in combination using the androgen receptor transactivation assay and MTT cell proliferation assay described in Example 1.

ASCJ-15 was administered at a concentration of 0, 0.5 or 1.0 μM in the androgen receptor transactivation (AR) assay as described in Example 1. Zhu et. al. previously demonstrated that SB is able to inhibit transcriptional activity using a concentration as little as 50 μM. In the present invention, SB was provided at 0 or 20 μM. Zhu et. al., Silymarin inhibits function of the androgen receptor by reducing nuclear localization of the receptor in the human prostate cancer cell line LNCaP. Carcinogenesis 22: 1399-1403, 2001. Referring to FIG. 6, ASCJ-15 and SB, alone and in combination, were capable of suppressing DHT-induced androgen receptor (AR) activity in LNCaP cells. When administered in combination, ASCJ-15 and SB suppressed 70% of DHT-induced androgen receptor activity in an additive fashion. 70% suppression of DHT-induced activity could also be calculated when combining the suppressive effect of 20 μM SB and 1 μM of ASCJ-15, administered alone.

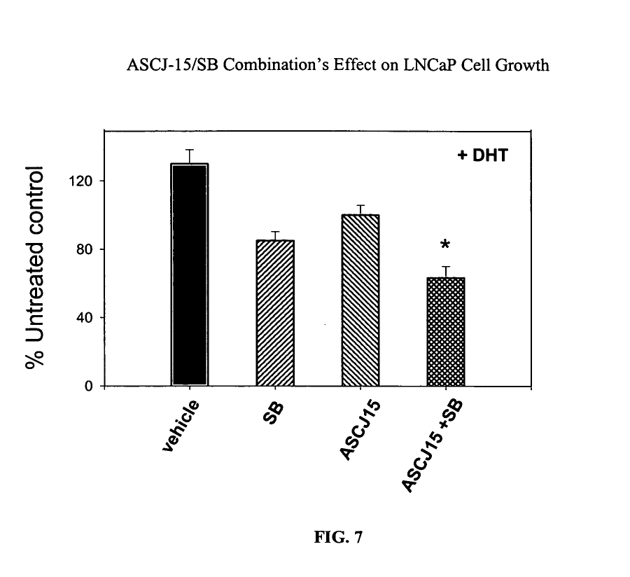

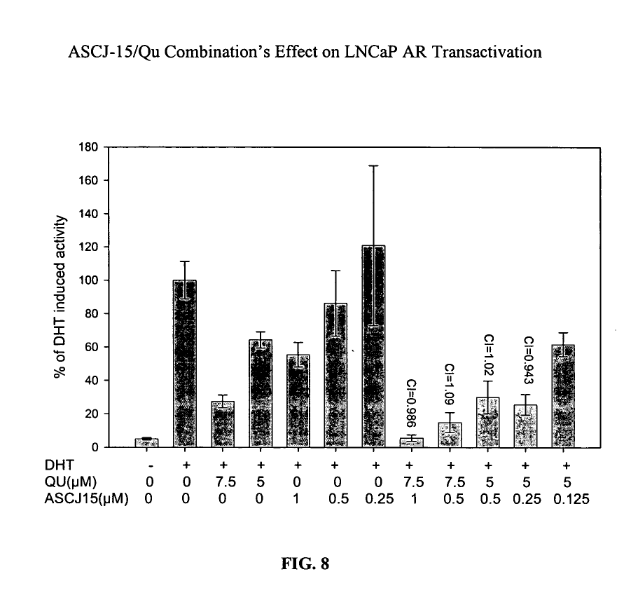

ASCJ-15 and SB were also tested alone and in combination for the efficacy in control of human prostate tumor cell growth. LNCaP cells were used in the MTT assay as described in Example 1. The results are depicted in FIG. 7. ASCJ-15 and SB were capable of suppressing tumor cell proliferation alone and in combination. When administered in combination, ASCJ-15 and SB suppressed tumor cell proliferation additively.

### Example 6

### Inhibiting Steroid-induced Gene Activation by Administering a Compound Capable of Inducing Degradation of a Steroid Receptor in Combination with a Compound Capable of Suppressing Transcription of the Steroid Receptor mRNA

The effect of a compound capable of inducing degradation of an androgen receptor (AR) was administered alone and in combination with a compound capable of suppressing transcription of the androgen receptor. ASCJ-15 as demonstrated in Example 2 is capable of degrading the androgen receptor. A purified form of Quercetin (QU), a flavonoid found in apples, onions and green tea, is believed to suppress transcription of the androgen receptor mRNA. Xing et. al., Quercetin inhibits the expression and function of the androgen receptor in LNCaP prostate cancer cells. Carcinogenesis 22: 409-414, 2001. Each were administered alone and in combination using the androgen receptor transactivation assay described in Example 1.

ASCJ-15 was administered in concentrations of 0, 0.125, 0.25, 0.5 or 1.0 μM in the androgen receptor (AR) transactivation assay described in Example 1. Quercetin (QU) was administered at 0, 5 or 7.5 μM. Referring to FIG. 8, both ASCJ-15 and QU were capable of suppressing DHT-induced androgen receptor (AR) activity. When administered in combination, the two test compounds suppressed DHT-induced activity synergistically.

### Example 7

### Inhibiting Steroid-induced Gene Activation by Administering a Compound Capable of Inducing Degradation of a Steroid Receptor in Combination with a Compound Capable of Inhibiting Steroid to Steroid Receptor Binding

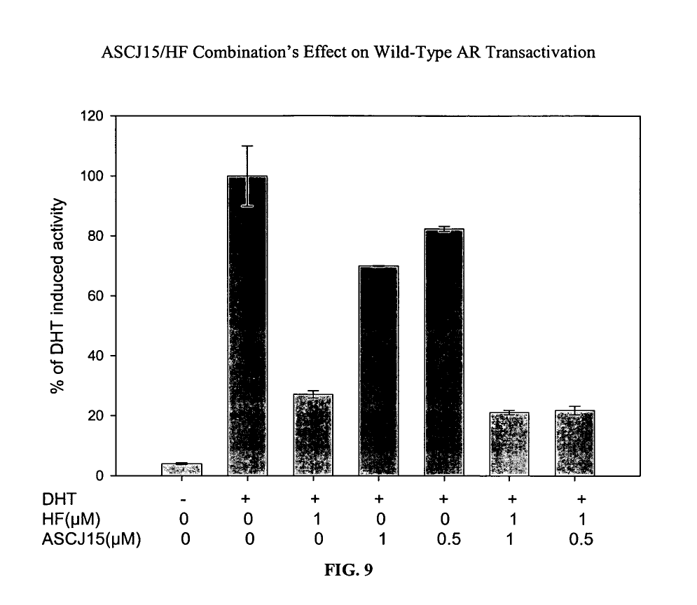

The effect of a compound capable of inducing degradation of an androgen receptor (AR) was administered alone and in combination with a compound capable of inihibiting binding between androgen and the corresponding androgen receptor (AR). ASCJ-15 as demonstrated in Example 2 is capable of degrading the androgen receptor (AR). Hydroxyflutamide (HF), a metabolite of flutamide is believed to block binding between androgen and the androgen receptor (AR). HF was tested alone and in combination with ASCJ15 in the androgen receptor transactivation assay described in Example 1. Each compound was tested for the ability to suppress the activity of a wild type androgen receptor and a mutant androgen receptor using LNCaP tumor cells. HF and ASCJ15 alone and in combination suppressed wild type androgen receptor (AR) activity. When administered in combination the suppression was additive, as depicted in FIG. 9.

All headings are for the convenience of the reader and should not be used to limit the meaning of the text that follows the heading, unless so specified. All documents referred to or cited are incorporated by referenced in their entirety.

1. A method of inhibiting or reducing steroid-dependent gene activation comprising:
a) providing a biological sample comprising a steroid receptor;
b) administering a first compound to said biological sample, wherein said first compound is capable of inducing degradation of said steroid receptor; and
c) administering a second compound to said biological sample, wherein said second compound is capable of inhibiting said steroid receptor from activating a gene;
wherein said first compound and said second compound act at different steps within a gene activation pathway of said steroid receptor;
further wherein steroid-dependent gene activation is reduced or inhibited greater than if said first compound or said second compound are administered alone.

2. The method according to claim 1, wherein said steroid receptor is an androgen receptor.

3. The method according to claim 1, wherein said first compound comprises a curcumin derivative or analog.

4. The method according to claim 2, wherein said curcumin derivative is selected from the group consisting of ASCJ-9, ASCJ-15 and a functional derivative thereof.

5. The method according to claim 1, wherein said second compound is able to inhibit, in part, a steroid binding to said steroid receptor.

6. The method according to claim 5, wherein said second compound is selected from the group consisting of a bicalutamide, a hydroxyflutamide and a functional derivative thereof.

7. The method of claim 1, wherein said second compound is able inhibit, in part, a steroid receptor binding to a steroid response element.

8. The method of claim 7, wherein said second compound is docosahexaenoic acid (DHA) or a functional derivative thereof.

9. The method according to claim 7, wherein said steroid response element is an androgen response element (ARE).

10. The method according to claim 1, wherein said second compound is able to inhibit, in part, nuclear transfer of said steroid receptor.

11. The method according to claim 10, wherein said second compound is silibinin (SB) or a functional derivative thereof.

12. The method according to claim 1, wherein said second compound is able to inhibit, in part, transcription of said steroid receptor.

13. The method according to claim 12, wherein said second compound is quercetin (QU) or a functional derivative thereof.

14. The method according to claim 1, wherein said first compound and said second compound are administered together.

15. A composition comprising:
a first compound capable of inducing degradation of a steroid receptor; and
a second compound capable of inhibiting activation of a gene by said steroid receptor, wherein said second compound does not significantly induce degradation of said steroid receptor.

16. A pharmaceutical composition comprising:
a) a first compound or pharmaceutically acceptable salt thereof able to induce degradation of a steroid receptor;
b) a second compound or a pharmaceutically acceptable salt thereof able to inhibit gene activation by said steroid receptor; and
c) a pharmaceutically acceptable diluent, adjuvant or carrier; and
wherein said first compound and said second compound, when in combination, are provided in a therapeutically effective amount.

17. The pharmaceutical composition according to claim 16, wherein said first compound is a curcumin derivative or analogue.

18. The pharmaceutical composition according to claim 16, wherein said second compound or pharmaceutically acceptable salt thereof is selected from the group consisting of bicalutamide, hydroxyflutamide, docosahexaenoic acid (DHA), silibinin (SB), vitamin E succinate (VES), quercetin (QU), finesteride, dutasteride, or a functional derivative thereof.

19. The pharmaceutical composition according to claim 16, wherein said first compound and said second compound are encapsulated in a liposome.

20. A method of preventing or treating a steroid modulated medical condition in a human comprising:
a) providing an individual suspected of suffering from a steroid modulated medical condition; and
b) administering a therapeutically effective amount of the pharmaceutical composition according to claim 16 to said individual.

21. The method according to claim 20, wherein said steroid modulated medical condition is selected from the group consisting of acne, hirsutism, androgenetic alopecia (male pattern baldness), prostate cancer, benign prostate hyperplasia, bladder cancer, liver cancer, breast cancer, cervical cancer and lung cancer.

22. A method of treating a wound or inflammation comprising:
a) providing an individual having a wound site or inflammatory site; and
b) topically administering the pharmaceutical composition according to claim 16 to said wound site or inflammatory site.

23. A method of inhibiting or reducing steroid-dependent gene activation in a human comprising:
a) providing the pharmaceutical composition of claim 16; and
b) encapsulating said pharmaceutical in a liposome;
c) administering said encapsulated pharmaceutical to a human.
